(19) United States
(12) Patent Application Publication
(10) Pub. No.: US 2008/0156896 A1
(43) Pub. Date: Jul. 3, 2008

(54) Spray Device

(75) Inventors: James Anderson, Hull (GB); Wu Jin, Hull (GB); Simon Woolley, Hull (GB); Ivan Ye, Hull (GB)

Correspondence Address: Andrew N. Parfomak;Norris McLaughlin & Marcus, PA, 875 Third Avenue, 18th Floor, New York, NY, 10022, US

(73) Assignee: RECKITT BENCKISER (UK) LIMITED, Slough (GB)

(21) Appl. No.: 11/816,077

(22) PCT Filed: Feb. 2, 2006
(86) PCT No.: PCT/GB2006/000345

§ 371 Date: Oct. 30, 2007

(30) Foreign Application Priority Data
Feb. 15, 2005 (GB) 0503098.6

(51) Int. Cl. A61L 9/04; B67D 5/08; B67D 5/00

(52) U.S. Cl. 239/34; 222/52; 222/153.14

(57) ABSTRACT

The invention relates to a spraying device (10) comprising a material container (16), a control circuitry (24) and an outlet section (22), wherein the control circuitry (24) includes an electromagnetic switch (26) that is operable to allow the control circuitry (24) to control the outlet section (22) only in the presence of a magnet (28) associated with the material container.

This invention relates to spray device, particularly but not exclusively, an electronically controlled spray device for use with a pressurised container.

Existing types of electronically controlled spray devices include those which include timed activation of a lever arm which is caused periodically to press down onto a dispensing head of an aerosol container. Pressing down on the dispensing head causes ejection of material from the aerosol canister and into the atmosphere. Such devices are used for dispensing air freshening and deodorising formulations.

Problems with devices of this type arise in that different formulations are often used for different countries, to account for local legislation and/or local preferences. It is disadvantageous to use the same spray device with different formulations, because there are different requirements as to dose of material between the different formulations referred to above. Thus, the same volume of one formulation dispensed into a room will have a different effect to the same volume of different formulation.

According to a first aspect of the present invention, there is provided a spraying device comprising a material container, control circuitry and an outlet section, wherein the control circuitry includes an electromagnetic switch that is operable to allow the control circuitry to control the outlet section only in the presence of a magnet associated with the material container.

The electromagnetic switch is preferably a reed switch. The outlet section preferably includes a solenoid. The material container is preferably a pressurised container, such as an aerosol canister. The spraying device preferably includes a power supply.

The material container is preferably held in a sleeve, which sleeve preferably holds the magnet. The sleeve may be secured in an opening in the spraying device. The sleeve preferably ensures a desired separation between the magnet and the electromagnetic switch.

The outlet section is preferably axially aligned with an outlet portion, or outlet stem, of the material container.

The spraying device preferably comprises a body section of which the power supply, control circuitry, outlet section and electromagnetic switch may form a part. The body section may be a chassis.

The body section may be adapted to receive a cover portion. The output section is preferably adapted to direct material through an opening in the cover portion. The cover portion may be replaceable.

The control circuitry may include level determining means operable to determine a level of material in the material container. The level determining means may incorporate a counter operable to count a number and duration of activations of the outlet section for comparison to a known value for a given material container. The level determining means may be operable to determine an amount of work done by the outlet section against a material held in the material container, an amount of work done being higher when material remains in the material container when pressurised, in the latter case the level determining means is preferably operable to detect when an amount of work falls below a predetermined threshold. The level determining means may be operable to determine differences in a sound of material passing through the outlet section for different levels of material in the material container. The level determining means may be operable to detect a change in light transmitted across a flow path of material from the material container to determine changes in the amount/presence of material in the flow path and thus a level of material in the material container.

Preferably, the level determining means are operable to use multiple detections to assess a level of material in the material container, to advantageously increase an accuracy of level determined.

The body section preferably includes a power supply housing. The housing is preferably closed at an underside of the body section, preferably by a resilient clip that is adapted to engage the body section. The power supply housing is preferably substantially separate from a sleeve-receiving section. The power supply housing is preferably free standing with respect to the sleeve-receiving section. A web section may join an upper part of the power supply housing with the sleeve-receiving section.

The body section preferably includes one of a projection and recess pair, the other of the projection and recess being part of the sleeve of the material container. The projection and recess pair preferably being adapted to retain the sleeve in the body section. Preferably the recess is in the body section, preferably in the form of an opening, preferably a pair of openings, preferably on opposite sides of the sleeve receiving section of the body section. The projection and recess pair are preferably adapted to retain the sleeve in a position that causes an outlet stem of a pressurised container held in the body section to be depressed. In such a situation the valve stem is preferably pushed, in use, into or against a sealing assembly, which sealing assembly preferably forms part of the outlet section.

The sleeve receiving section may include sleeve locating means, which may be adapted to receive sleeve locating means of the sleeve. The sleeve locating means may comprise a projection and recess pair. Preferably the sleeve locating means of the sleeve receiving section comprise a groove, which groove preferably has a rounded profile and preferably is aligned with a longitudinal axis of the sleeve receiving means. The sleeve locating means of the sleeve receiving section preferably extend above a base section of the sleeve receiving section to below a head section thereof.

The body section preferably includes an outlet section holding portion, preferably on the head section of the body section. The outlet section holding portion preferably includes resilient holding means for the outlet section, said resilient holding means preferably including at least one tab element, preferably two tab elements, adapted to be located on opposite sides of the outlet section.

According to another aspect of the present invention there is provided a spraying device comprising a power supply, control circuitry and an outlet section forming a body section, the body section being adapted to receive a material container, wherein the body section is adapted to receive a removable cover section.

The removable cover section preferably has an outlet opening, preferably on an upper face thereof, said upper face is preferably concave. Advantageously, the concave, or dished, face assists in the dispersal of material by allowing a broader spray cone than would be possible with a flat upper face.

The opening, outlet section and material container are preferably substantially coaxially arranged.

According to another aspect of the invention there is provided a spraying device body section comprising control circuitry and an outlet section, the body section incorporating a material container receiving section being adapted to receive a material container, the body section being further adapted to receive a replaceable cover section.

The material container is preferably receivable in an opening in the body section, which opening is in a lower face of the body section.

The outlet section is preferably located above the material container receiving section. The outlet section is preferably substantially coaxial with the material container receiving section. The outlet section is preferably separated from the material container receiving section along a common longitudinal axis of the outlet section and material container receiving section.

The spraying device preferably incorporates mounting means, which are preferably adapted to mount the spraying device on a wall or other vertical surface. Preferably, the mounting means are adapted to allow mounting of the spraying device so that material is ejected from the spraying device at an angle away from the mounting surface.

Preferably, in use, an edge of a spray cone of material from the spraying device, which edge is closest to the mounting surface, is either parallel with or angled away from the mounting surface.

According to another aspect of the invention there is provided a spraying device comprising a body section comprising control circuitry, an outlet section and a power supply holding section, wherein the body section is adapted to receive a material container held in a sleeve and wherein the body section forms a base of the spraying device.

The sleeve is preferably retained entirely within the body section.

All of the features described herein may be combined with any of the above aspects, in any combination.

For a better understanding of the invention, and to show how embodiments of the same may be carried into effect, reference will now be made, by way of example, to the accompanying diagrammatic drawings in which:

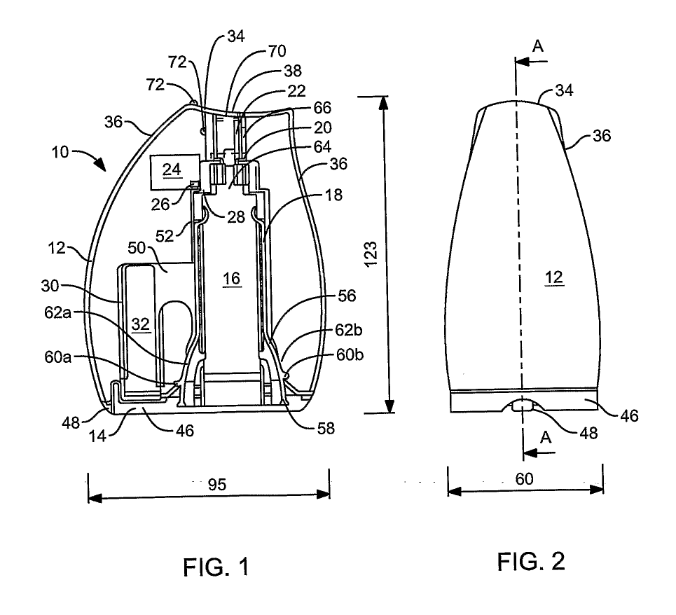

FIG. 1 is a schematic cross-sectional side view of a spray device comprising an outer cover, a chassis and a replaceable aerosol canister held in a sleeve;

FIG. 2 is a schematic perspective front view of the spray device;

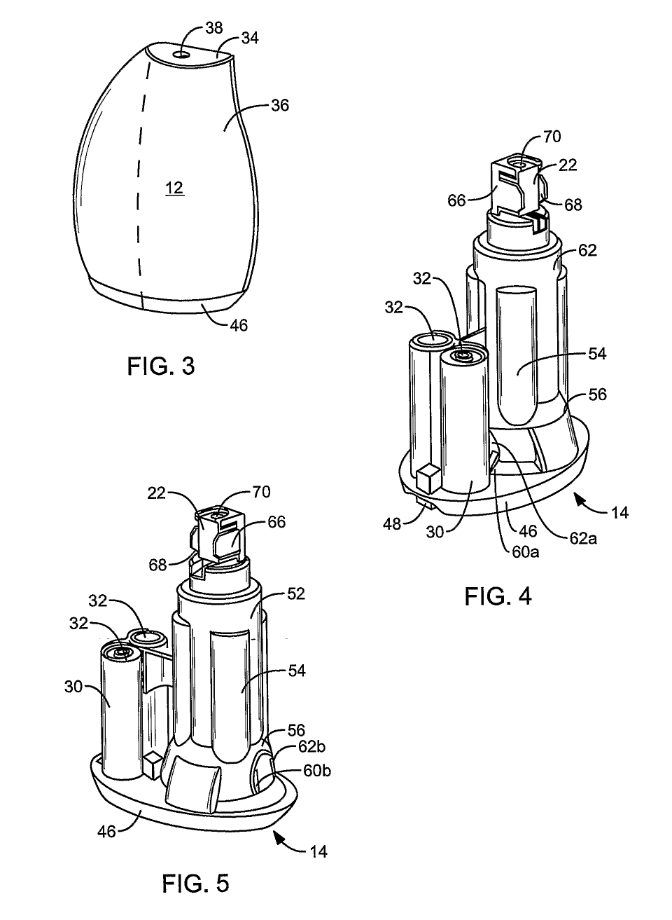

FIG. 3 is a schematic perspective view of the spray device;

FIG. 4 is a schematic perspective view from the front and to one side of the spray device chassis;

FIG. 5 is a schematic perspective view from the side of the chassis;

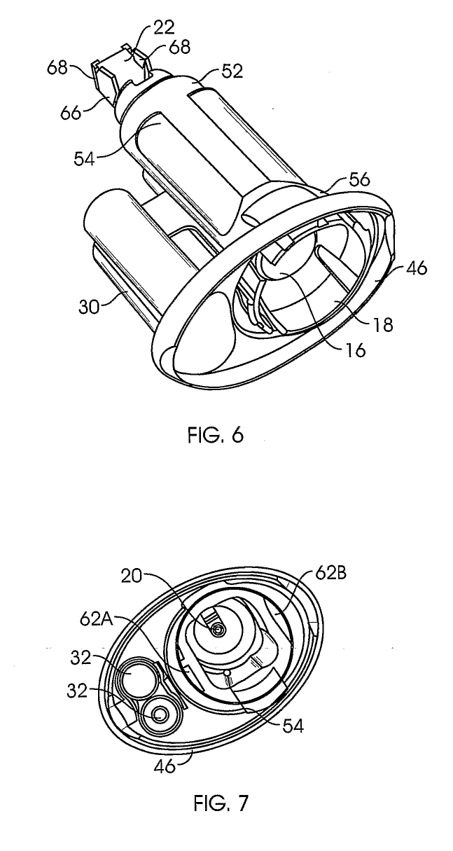

FIG. 6 is a schematic perspective view from below of the chassis incorporating the aerosol canister in its sleeve;

FIG. 7 is a schematic perspective view from below of the chassis without the sleeve and aerosol canister;

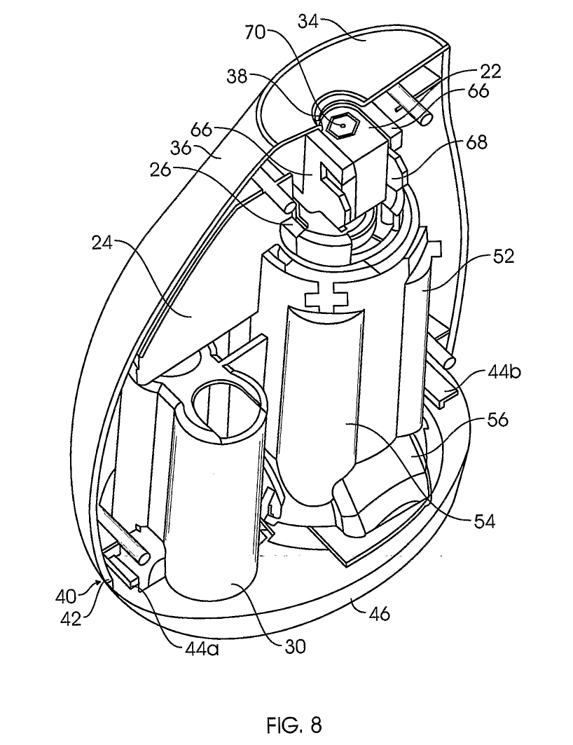

FIG. 8 is a schematic partially cut away perspective view of the spray device;

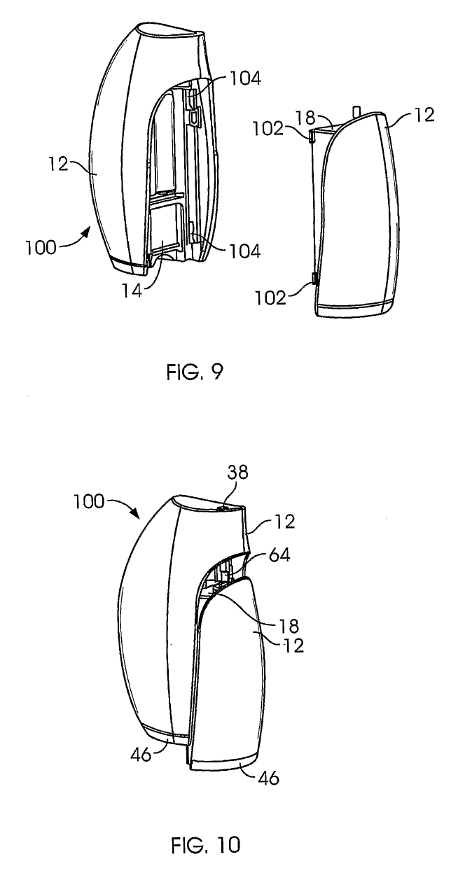

FIG. 9 is a schematic perspective partially exploded view of a second embodiment of spray device;

FIG. 10 is a schematic perspective view of the spray device showing a removable aerosol canister section partly engaged with an outer cover and chassis section of the spray device;

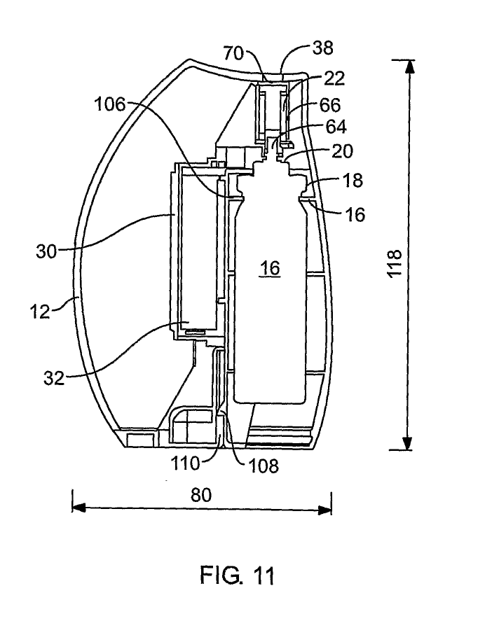

FIG. 11 is a schematic cross-sectional side view of the second embodiment of spray device; and

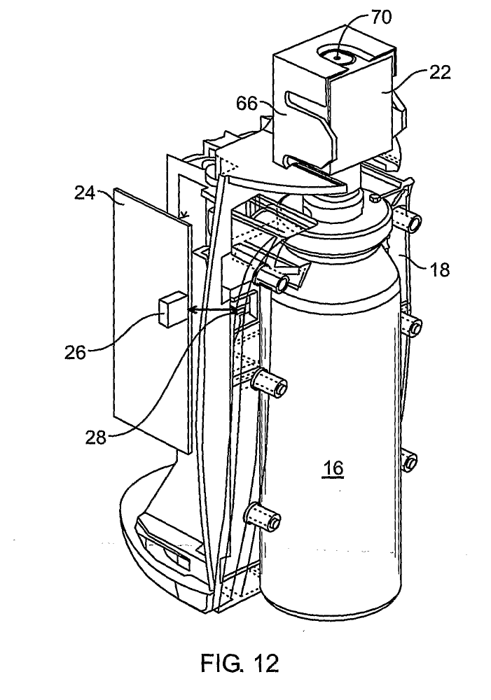

FIG. 12 is a partial schematic perspective view of part of the chassis and aerosol canister sleeve of the second embodiment.

Aerosol spray devices are popular for dispersing fragrances, deodorising preparations, sanitising preparations and other materials. Such spray devices are becoming more complex as more control over the amount, timing and release of the material in the aerosol container is provided. With this increasing complexity comes a need for greater control over replacement aerosol canisters for the spray devices. The need for greater control arises because damage to the fragile stem of an aerosol canister can lead to malfunctioning of the spray device. The spray devices described herein have significant advantages in that they accept only aerosol canisters that are held in a particular sleeve that is more fully described in the applicant's co-pending 30 application. The spray devices described herein have been designed in order to minimise the risk of damage to a stem of the aerosol canister which is to be placed in the spray device. Also described is a spray device chassis which is designed to receive the above-mentioned sleeve and aerosol canister, as well as receiving an outer cover which can be modified for subsequent models issued by the manufacturer whilst the chassis remains the same. For example colours or surface features of the outer cover may be changed.

An aerosol spray device 10 comprises an outer removable shell 12, an internal chassis 14 and an aerosol canister 16 held within a sleeve 18.

The internal chassis 14 includes a sealing assembly 20 comprising a face seal and an O-ring and is described in a further co-pending application. The internal chassis 14 also includes a magnetically operated valve, such as a solenoid switch or solenoid valve 22 that is controlled by control circuitry on a printed circuit board (PCB) 24. A reed switch 26 is located on the chassis 14, which reed switch 26 functions with a magnet 28 located on the sleeve 18. The control circuitry on the PCB 24 controls operation of the solenoid switch 22 according to programmable features such as spray dose, controlled by the length of time that the solenoid switch is opened at a given time, and spray interval.

The internal chassis 14 also includes a battery housing 30 for batteries 32 which are used to power the PCB 24 and solenoid switch 22.

In more detail, the outer removable shell 12 is made of a plastics material that can be mouldable to a suitable shape that is visually attractive. The outer contours of the shell are generally curved, except for a top section 34 that is dished and has angular edges where it joins side walls 36 of the shell. An opening 38 is provided in the top section 34 of the removable shell 12, which opening 38 communicates with the solenoid switch 22 to allow the ejection of material from the aerosol canister 16. A lower rim 40 (see FIG. 8) of the shell 12 may incorporate a bead 42 that is arranged to clip under tabs 44a and 44b of the internal chassis 14 to ensure engagement of the removable shell 12 with the internal chassis 14. The outer cover is preferably in two parts 12a and 12b (see FIG. 2) that are held together by screws (not shown). The colour of the plastics material from which the outer removable shell 12 is formed can be chosen to correspond to a fragrance or type of fragrance or be inspired by the type of fragrance that is held within the aerosol canister 16.

The internal chassis 14 forms the lowest part of the aerosol spray device 10, so that a lower section 46 thereof forms a base of the aerosol spray device 10. The spray device 10 is designed to stand on the lower section 46, with the sleeve 18 held within.

As seen in FIG. 1, a clip 48 is provided at a base of the battery housing section 30 of the internal chassis 14. Which clip, when pushed to the right, as shown in FIG. 1 will release, pivot down and allow access to the batteries 32. A web 50 connects an upper part of the battery housing section 30 with a sleeve receiving section 52 of the chassis. The web 50 and an upper part of the battery housing section 30 may provide support for the PCB 24 in the event that it extends down to that level, unlike the PCB 24 shown in FIG. 1 which is much smaller. The battery housing section 30 is free-standing, except for the connection with the web 50. The web 50 may have an opening (not shown) therein to allow the screw(s) holding the outer shell 12 together to pass therethrough. This feature assists structural stability of the outer shell 12 in relation to the chassis 14.

The sleeve receiving section 52 forms a tubular section in which the sleeve 18 is received, as shown in FIG. 1. The sleeve receiving section 52 incorporates (when viewed from the outside) a locating rib 54, which, when viewed from the inside of the hollow interior of the sleeve receiving section 52 forms a locating groove 54 (see FIG. 7), which groove 54 matches and receives a corresponding rib on the sleeve 18. The combination of the groove 54 of the sleeve receiving section 52 and the rib of the sleeve 18 (both of which have the same rounded profile) allows location of the sleeve 18 within the sleeve receiving section 52 of the internal chassis 14 in only one orientation about a longitudinal axis of the sleeve. This ensures that the magnet 28 held on the sleeve 18 is in the desired position, close to the reed switch 26 on the PCB 24.

A lower flared section 56 of the sleeve receiving section 52 corresponds to a lower flaring 58 of the sleeve 18. As shown in FIGS. 1, 4 and 5 projections 60a/60b of the lower flaring 58 project through openings 62a/b in the lower flared section 56. The projections 60a/60b engage by a snap fit in the lower openings 62a/b to ensure location of the sleeve 18 and also retention of the sleeve 18 within the internal chassis 14.

The combination of the locating rib of the sleeve 18 and the locating groove 54 of the internal chassis 14, together with a close fit of the sleeve 18 within the sleeve receiving section 52 ensure that the output stem 64 of the aerosol canister 16 approaches the sealing assembly 20 of the solenoid switch 22 at, or close to, an angle perpendicular with the sealing face of the sealing assembly 20. The angle of approach is particularly important to ensure that damage to the sealing assembly 20 by the output stem 64 is minimised. It is desired to minimise such damage, because leaking of material from within the aerosol canister 16 could result if the sealing assembly 20 or the output stem 64 are damaged. In the event that non-compatible seals are used, there is potentially a fire risk from material held within the aerosol canister 16.

The use of the sleeve 18 for the aerosol canister 16 ensures the proper approach of the output stem 64 of the aerosol canister to the sealing arrangement 20. In addition, use of the sleeve 18, from which the aerosol canister 16 cannot be removed without damaging the sleeve 18, ensures that only aerosol canisters containing known formulations are used with the internal chassis 14 to spray material from the aerosol canister 16. The control of which formulations are used with the aerosol spray device 10 is particularly important in relation to the proper functioning of the aerosol spray device 10. There are also safety issues, as mentioned above.

An upper section 66 of the internal chassis 14 holds the solenoid switch 22 in position in a snap fitting, in which tabs 68 of the upper section 66 part slightly on insertion of the solenoid switch 22 into the upper section 66 and then snap closed on full insertion of the solenoid switch 22. The solenoid switch incorporates the sealing assembly 20 into which the output stem 64 of the aerosol canister 16 is inserted, as mentioned above. The solenoid switch 22 is controlled by the PCB 24 (not shown in FIGS. 4 and 5 for clarity, but shown in FIG. 1 and FIG. 8).

The solenoid switch 22 will only function when the reed switch 26 is closed, which in turn will only close in the presence of the magnet 28 which is present on the sleeve 18. In this way, the aerosol spray device 10 will only function in the presence of an authorised sleeve 18 bearing a magnet. This has advantages which follow on from those mentioned above in relation to the use of only sleeves 18 and hence aerosol canisters 16 that have been approved by the manufacturer, or other legitimate party, of the aerosol spray device 10.

Signals from the PCB 24 control the operation of the solenoid switch 22. Signals include those of opening and closing the solenoid switch 22 to allow ejection of material from the aerosol canister 16. The duration of opening of the solenoid switch 22 and periods in between successive openings are controlled by the PCB 24. It is the solenoid switch 22 that controls output of material from the aerosol canister 16, because by placing the aerosol 16 in the sleeve 18 and the sleeve into the internal chassis 14 the output stem 64 of the aerosol canister is depressed causing release of material from the aerosol canister 16. However, the sealing assembly 20 prevents material escaping from the solenoid switch 22 in the event that the solenoid switch 22 is closed. The provision of control of ejection of material from the aerosol canister has significant advantages over the release of material from the aerosol canister 16 simply by depression of the output stem 64 in the usual way, because greater control can be arrived at, as described below.

When the solenoid switch 22 is open material is ejected from an upper opening 70 in the solenoid switch 22.

The PCB 24 incorporates a timer, which has a clock to allow for setting of the activation of the solenoid switch 22 at certain times of day.

In use, the solenoid switch 22 can be activated in the following ways. A general pulse spray is used, which may have a duration of 1 ms to 200 ms, preferably 5-30 ms. Also, there is a boost operation which opens the solenoid switch 22 for approximately two to ten, preferably three to seven, or less or more, times longer, for example 40 ms to 200 ms. Of course there is sufficient variability in the solenoid switch 22 that continuous spraying from the aerosol canister 16 could be achieved if desired until the aerosol canister 16 is completely exhausted, which may take approximately 30 seconds. The options for a continuous spray and boost facility as referred to above from the same reservoir are particularly advantageous, especially when compared to devices that use a heating element to disperse a fragrance into a room or require a fan device, both of the former requiring considerably higher power than is the case with the solenoid switch 22 used in the device described herein. Additionally, the mechanical depression of an aerosol canister valve stem using a motor driving a cog mechanism which uses considerably more power than the system described herein and so uses batteries more quickly, with obvious disadvantages in relation to cost and efficiency. Also, the use of a motor gives rise to excessive noise.

A mounting plate 94 is provided on an outer side of the spraying device on the part 12a of the outer shell. The mounting plate 94 may be adhesive backed and is angled so that the opening 38 in the top section 34 of the shell 12 is angled away from the vertical, so that, in use, a spray cone 96 of material is ejected away from a wall to which the device is attached.

The control of the intermittent spray function is controlled by a user by use of a slide switch 90 (see FIG. 3), which can be used to select three settings, for example every 5 minutes, every 10 minutes or every 20 minutes, as desired. The boost is operated by a switch 92.

On initial insertion of the sleeve 18 into the internal chassis 14 the reed switch 26 is closed. This may provide a trigger to the PCB 24, which could also be provided by a re-set switch (not shown) of the PCB 24. These signals can be used to ensure that a first activation of the aerosol canister 16 and first ejection of material therefrom is in the boost mode, to ensure that gas that is normally held at the top of a aerosol canister 16 after manufacture is issued first is completely exhausted to allow the liquid fragrance held within the canister 16 to be properly ejected. Also, when an aerosol spray device 10 is first placed in a room, it will be necessary to build up a concentration of the fragrance held within the aerosol canister 16 in the room. Thus, the first few ejections may be on the boost setting to ensure a more rapid dispersal of the fragrance in the room.

Control over the solenoid switch 22 may be provided by sensors connected to the PCB 24. Such sensors may include light sensors, to ensure that fragrance is only ejected when needed, i.e. when light is detected. Motion sensors may also be used, which could ensure that fragrance is only ejected when movement is detected, i.e. when people are in the room. A timing clock having functionalities similar to that in central heating system control clocks may be provided on the internal chassis 14 to allow setting of a weekly/daily program of ejection of fragrance into a room. The PCB 24 may also be operable to receive signals by means of suitable circuitry on the PCB 24 from wireless devices, such as mobile phones via SMS or Bluetooth transmission. Also, wireless signals from computers may be received, by the PCB 24 to effect a control of the aerosol spray device 10.

An indication device, such as a visual indication in the form of an LED 72 (shown only in FIG. 1) is provided on the outer removable shell 12. The LED may be used to indicate by means of a double flash that the aerosol spray device 10 is about to eject material from the opening 38 in the outer shell 12. Alternatively, the LED 72 may be located on the internal chassis 14 and may be arranged to allow light from the LED 72 to pass through the shell 12 so that it can be seen by a user. A pre-event communication such as the LED 72 may also be provided by a sound or a vibration, as desired.

The visual indicator may also be used to indicate when the aerosol canister 16 is at or approaching an empty state. The indication used may be, for example, for the LED 72 to flash every second when there is approximately 5-10% of the material remaining in the aerosol canister 16. The LED 72 may flash every half second when an aerosol canister is between approximately 5% full and empty. The latter flashing may last for approximately 2 days, after which flashing will cease in order to maintain battery life. Detection of the empty state could be inferred by counting a number of activations of the solenoid switch 22, taking into account the duration of each of the activations, which can be stored. A total number of activations of a given time could be pre-programmed into the PCB 24 for a typical aerosol canister 16, which could form the basis of the calculation of the end of life.

Other methods by which the PCB 24 may determine an end of life of the aerosol canister 16 include determination of work done by the solenoid switch 22 against material being ejected from the aerosol canister 16 under pressure. When the aerosol canister 16 is exhausted less work will be required from the solenoid switch 22 to close. The smaller amount of work done will be detectable by the solenoid switch 22 drawing less current from the PCB 24, which reduction in current could be used to infer that the aerosol canister 16 is exhausted. The PCB 24 will incorporate a threshold value that is required for current drawn by the solenoid switch 22 in usual spray operation. When the current falls below this value, an indication of either a low level of material in the aerosol canister or no material in the aerosol canister 16 is indicated.

Another method of determining the end of life is by detection of the sound that the solenoid switch 22 makes, which will differ between spraying material from the aerosol canister 16 and the sound made when no material is left in the aerosol canister 16. Suitable detection of the difference between the sounds can be used to indicate that the aerosol canister 16 is running out of material by means of a visual or audible indication as mentioned above.

A further method of detecting the end of life in the aerosol canister 16, is by use of a light sensor across a flow path through the solenoid switch 22. Detecting differences in the amount of light transmitted between a situation when there is material in aerosol canister to eject and the situation in which there is no material can be used to indicate that the aerosol canister 16 is approaching or at an end of life.

The device described above provides significant advantages in that the aerosol spray device delivers real measurable, both by weight of material ejected and by a user perception, adjustability without the need for power delivered by a fan or heater or a combination thereof. The pulse delivery system described above for the aerosol canister 16 is capable of optimising the dose and frequency to deliver a continuous and consistent perception of fragrance to a user. This may be by ejecting a greater amount of material at certain times to improve the perception to a user, as described above. The device may also be made without the reed switch/magnet pair, whilst still gaining some or all of the benefits mentioned above.

FIGS. 9 to 12 show an alternative embodiment of aerosol spray device 100. The second embodiment 100 has much in common with the first embodiment described above and much of the same functionality and features. Where like features are present, like numerals have been used.

The main differences to the first embodiment are that the aerosol canister 16 is held in a sleeve, part of which sleeve forms a section of the outer shell 12. The sleeve 18 with a portion of the outer shell 12 engages the remainder of the shell 12 and the internal chassis 14 by means of projections 102 of the sleeve 18 being received in corresponding opening/grooves 104 of the chassis 14.

The sleeve 18 differs in comprising two sections which are secured together to hold the aerosol canister 16 between them by means of tabs 106 that press against the aerosol canister 16. This embodiment of the sleeve 18 is held in a position by a projection 108 of the sleeve being received on a projection 110 of the chassis 14. Removal of the sleeve 18 can be achieved by pressing the section incorporating the tab 108 to allow release from the projection 110.

The internal chassis 14 may alternatively incorporate a number of openings for a number of canisters 16, designed to allow a user to select one of a number of fragrances. Also a number of solenoid valves 22 may be provided, one for each canister 16, or only one solenoid 22 could be used, with a facility for changing which of the canisters 16 is to be used.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiments(s). The invention extends to any novel one, or any novel combination, of the features disclosed in this specification (including any accompanying claims, abstract and drawings), or to any novel one, or any novel combination, of the steps of any method or process so disclosed.

1. A fragrance, deodorising preparation or sanitising preparation spraying device comprising a material container, control circuitry and an outlet section, wherein the control circuitry includes an electromagnetic switch that is operable to allow the control circuitry to control the outlet section only in the presence of a magnet associated with the material container.

2. A spraying device as claimed in claim 1, in which the electromagnetic switch is a reed switch.

3. A spraying device as claimed in claim 1, in which the material container is held in a sleeve.

4. A spraying device as claimed in claim 3, in which the sleeve holds the magnet.

5. A spraying device as claimed in claim 3, in which the sleeve is secured in an opening in the spraying device.

6. A spraying device as claimed in claim 1, in which the outlet section is axially aligned with an outlet portion of the material container.

7. A spraying device as claimed in claim 1, which comprises a body section of which the power supply, control circuitry, outlet section and electromagnetic switch form a part.

8. A spraying device as claimed in claim 7, in which the body section is adapted to receive a cover portion.

9. A spraying device as claimed in claim 1, in which the control circuitry includes level determining means operable to determine a level of material in the material container.

10. A spraying device as claimed in claim 9, in which the level determining means incorporates a counter operable to count a number and duration of activations of the outlet section for comparison to a known value for a given material container.

11. A spraying device as claimed in claim 9, in which the level determining means are operable to determine an amount of work done by the outlet section against a material held in the material container.

12. A spraying device as claimed in claim 9, in which the level determining means are operable to determine differences in a sound of material passing through the outlet section for different levels of material in the material container.

13. A spraying device as claimed in claim 9, in which the level determining means are operable to detect a change in light transmitted across a flow path of material from the material container to determine changes in the amount/presence of material in the flow path and thus a level of material in the material container.

14. A spraying device as claimed in claim 7, in which the body section includes a power supply housing.

15. A spraying device as claimed in claim 7, in which the body section includes one of a projection and recess pair, the other of the projection and recess being part of the sleeve of the material container, wherein the projection and recess pair are adapted to retain the sleeve in the body section.

16. A spraying device as claimed in claim 15, in which the projection and recess pair are adapted to retain the sleeve in a position that causes an outlet stem of a pressurised container held in the body section to be depressed.

17. A spraying device as claimed in claim 7, in which the body section includes a sleeve-receiving section including sleeve locating means, which are adapted to receive sleeve locating means of the sleeve, the sleeve locating means comprising a rib and groove pair.

18. A spraying device comprising a power supply, control circuitry and an outlet section forming a body section, the body section being adapted to receive a material container, wherein the body section is adapted to receive a removable cover section.

19. A spraying device as claimed in claim 18, in which the removable cover section has an outlet opening on an upper face thereof.

20. A spraying device as claimed in claim 19, in which the outlet opening, outlet section and material container are substantially coaxially arranged.

21. A spraying device body section comprising control circuitry and an outlet section, the body section incorporating a material container receiving section being adapted to receive a material container, the body section being further adapted to receive a replaceable cover section.

22. A spraying device body section as claimed in claim 21, in which the material container is receivable in an opening in a lower face of the body section.

23. A spraying device body section as claimed in claim 21, in which the outlet section is located above the material container receiving section.

24. A spraying device body section as claimed in claim 21, in which the outlet section is substantially coaxial with the material container receiving section.

25. A spraying device body section as claimed in claim 21, in which the outlet section is separated from the material container receiving section along a common longitudinal axis of the outlet section and material container receiving section.

26. A spraying device body section as claimed in claim 21, which incorporates mounting means adapted to mount the spraying device on a wall or other vertical surface so that, in use, material is ejected from the spraying device at an angle away from the mounting surface.

27. A spraying device comprising a body section comprising control circuitry, an outlet section and a power supply holding section, wherein the body section is adapted to receive a material container held in a sleeve and wherein the body section forms a base of the spraying device.

28. A spraying device as claimed in claim 27, in which the sleeve is retained entirely within the body section.

29. (canceled)
